(19) United States
(12) Patent Application Publication
(10) Pub. No.: US 2026/0259165 A1
(43) Pub. Date: Sep. 3, 2026

(54) FLEXIBLE PASSIVE WIRELESS SENSOR FOR SPOILAGE DETECTION OF PACKAGED GOODS

(71) Applicant: Purdue Research Foundation, West Lafayette, IN (US)

(72) Inventors: Rahim Rahimi, West Lafayette, IN (US); Sarath Gopalakrishnan, Santa Rosa, CA (US); Jose Waimin, Gilbert, AZ (US)

(73) Assignee: Purdue Research Foundation, West Lafayette, IN (US)

(21) Appl. No.: 19/163,763

(22) PCT Filed: Mar. 11, 2024
(86) PCT No.: PCT/US2024/019460

§ 371 Date: Sep. 9, 2025

Related U.S. Application Data
(60) Provisional application No. 63/451,268, filed on Mar. 10, 2023.

(51) Int. Cl. G01N 27/22; G01N 33/12

(52) U.S. Cl. CPC G01N 27/221; G01N 33/12

(57) ABSTRACT

A pH sensor for determining food freshness may include a substrate and a working tag on the substrate. The working tag may have a first resonant frequency. A reference tag may also be on the substrate. The reference tag may have a second resonant frequency. The pH sensor may further include a first layer in contact with a portion of the working tag and not the second tag. The first layer may be water soluble. The pH sensor may include a second layer positioned on top of the first layer, the second layer configured to dissolve when exposed to an environment having a pH level higher than a threshold pH. Dissolution of the second layer may cause a change in the resonant frequency of the working tag.

### CROSS REFERENCE TO RELATED APPLICATIONS

This application claims the benefit of U.S. Provisional Application No. 63/451,268 filed Mar. 10, 2023, the entirety of which is hereby incorporated by reference.

### TECHNICAL FIELD

This disclosure relates to food waste and food safety and, in particular, to monitoring food with sensors

### BACKGROUND

Foodborne diseases cause a substantial burden on healthcare systems worldwide. Every year, 1 in 10 people fall ill due to foodborne diseases. These diseases account for one-third of deaths in the group of children under the age of 5, the loss of 33 million healthy life years, and an annual loss of 77.7 billion dollars. The root of food-borne diseases is the consumption of contaminated food and water. Sources of contamination include chemical and heavy metal pollution; however, bacteria, viruses, and parasites are the main drivers of disease.

To reduce the incidence of foodborne diseases, strict regulations on the collection, transportation, and storage of food products exist. A key element in preventing foodborne diseases is proper refrigeration. The USDA advises meat products must be cooked and maintained at 60° C. or above before serving or stored below 4.4° C. within two hours of preparation. The range between 4.4° C. and 60° C. is considered the “Danger Zone”, where the rapid proliferation of bacteria on the meat increases the chance of contracting a foodborne disease. Federal regulations require grocery stores to maintain packaged meat products 5-10 days under refrigeration at temperatures below 4.4° C. Although refrigeration is efficient in reducing food spoilage, pathogenic bacteria, such as L. monocytogenes, which account for 28% of foodborne related deaths, can grow at temperatures as low as −0.4° C. Also, studies on temperature variations within commercial and domestic refrigerators show temperatures can reach as high as 10° C. depending on location and light source. Even under ideal conditions, assuming uninterrupted refrigeration, there is a risk for contamination.

An early indicator of meat spoilage due to bacteria proliferation is the change in surface pH. The pH of slaughtered meats, including beef, chicken, and fish, is between 5.8 to 6.2. In most meat products, including chicken, bacteria decomposition leads to products such as methylamine, dimethylamine, and trimethylamine with high pH values around 10, which leads to an increase in the overall pH of the meat surface.

Traditional methods developed for monitoring meat pH and identifying spoilage include hand-held probes and pH test strips. These methods are limited to individual, invasive, labor-intensive testing which, in most cases, requires physical contact with the food product. This significantly limits the use of traditional methods for large-scale real-time monitoring of packaged foods.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates a top view of an example of a pH Sensor.

FIG. 2 illustrates a side view of an example of a pH sensor.

FIG. 3 illustrates a working principle of a pH sensor.

FIG. 4 illustrates an example of a system for detecting food spoilage.

FIG. 5 illustrates a manufacturing process for a pH sensor.

FIG. 6A-B illustrates the affect of the thickness of a passivation layer on various aspects of the pH sensor.

FIG. 7A-B illustrates scanning electron microscope (SEM) imaging and Fourier Transform Infrared Spectroscopy (FTIR) characterization of metalized Polyethylene Terephthalate (PET), laser etched PET, and pH sensitive coated tags.

FIG. 8A-B illustrates results from contact angle experiments.

FIG. 9A-B illustrates results from a scratch tape test.

FIG. 10A-D illustrates results from solubility characterizations.

FIG. 11A-B illustrate shifts in the resonant peak of working tags in buffers of various pH.

### DETAILED DESCRIPTION

Methods aimed towards wireless detection for the non-invasive assessment of surface pH have been developed. Several colorimetric sensors have been developed as wireless pH sensing platforms. Even though these designs are low-cost and scalable, major drawbacks occur due to the complexity of the reader and toxicity. Colorimetric sensors require visual assessment from the operator or package-by-package interrogation, which limits the scalability of the process. Synthetic pH-sensitive compounds such as bromocresol purple, methyl red, bromophenol blue, and chlorophenol red are associated with allergic and toxic reactions, which limits their use in food packaging applications.

RFID technology has also been developed for food packaging applications, which allows for wireless monitoring of flexible sensing tags. In these designs, changes to pH-sensitive coatings on a sensing coil are measured wirelessly represented by shifts in resonance frequency peaks and intensity. Most designs continue the use of PCBs and batteries for optimum sensitivity and communication. Although these designs offer stable communication and high sensitivity, they are limited due to high cost, complex fabrication, and risk of contamination relative to food packaging applications. Other designs aimed towards battery-less and chip-less wireless detection have been developed, but few have been designed for the purpose of monitoring pH in packaged food products. In one design, shifts in the resonant peak triggered by changes in swelling behavior of a pH-sensitive chitosan hydrogel coated onto an RFID are measured wirelessly. Although this battery-less chip-less design is sensitive to different pH values, it is difficult to distinguish between shifts at pH 4 and shifts at pH 10, which limits its use in spoilage detection. The reversibility of the sensor also makes it vulnerable to tampering. Furthermore, these designs are prepared by traditional methods of manufacturing flexible sensors such as inkjet and screen printing which are limited by ink cost, high sintering temperatures, and complex fabrication processes. Laser etching of metalized films has been identified as an alternative yielding highly conductive, robust, flexible sensors with modified surface properties.

In various embodiments of this disclosure, a battery-less, chipless, flexible pH sensor tag is manufactured through a laser etching process. Laser etching metalized polyethylene terephthalate (PET) films results in high wettability and improved adhesion by functionalizing the surface with metal nanoparticles in a one-step process. Sensor tags comprises a working and reference UHF resonator in pair coated with a pH-sensitive layer. The UHF resonators, capable of far-field communication for large area interrogation, have a distinct resonant peak in the GHz range, which is sensitive to changes in the coating thickness. The coating includes an S100 and PVA bilayer in which S100 is sensitive to changes in pH. Above a pH of 6.8, which indicates spoilage, S100 dissolves exposing the water-soluble PVA layer to the surrounding media, thus triggering a shift in the resonant peak. The coating thickness was optimized through simulations to achieve a minimum required shift of 20%. Solubility characterizations demonstrated the highly pH-dependent solubility of S100 and the rapid dissolution of PVA when exposed. The sensing tags achieved a distinct shift above 20% in buffers of pH>7 and were capable of detecting spoilage of refrigerated chicken samples packaged using standard materials. Additional and alternative embodiments and technical advancements are provided herein.

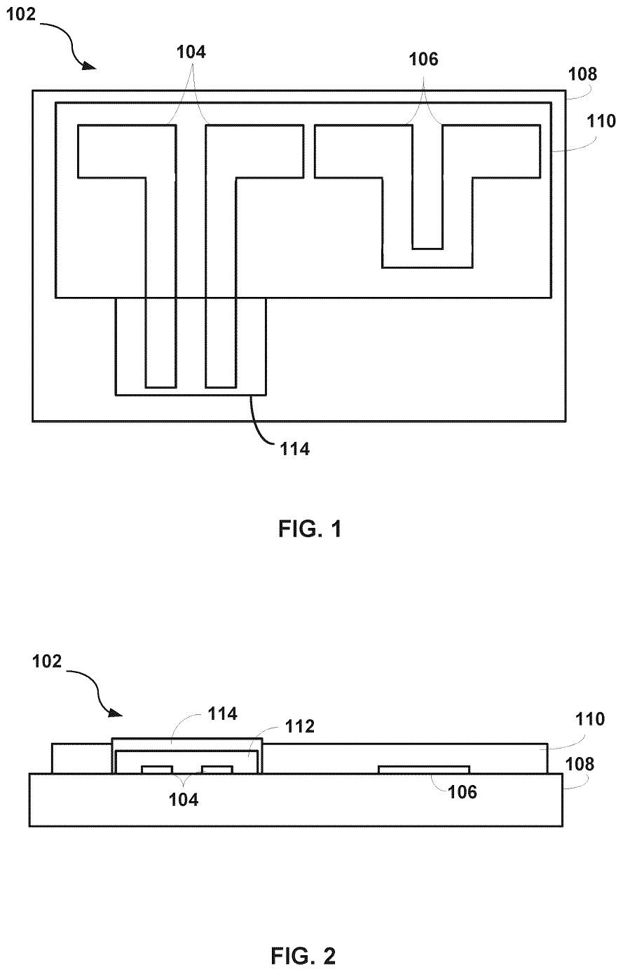

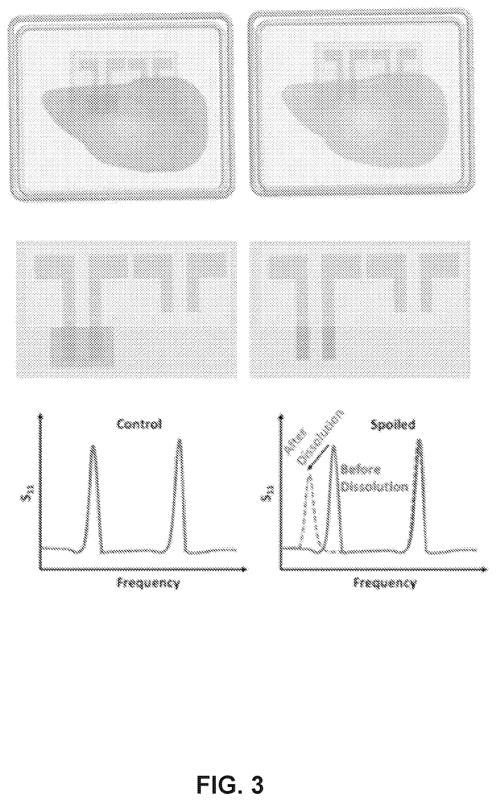

FIG. 1 illustrates a top view of an example of a pH Sensor 102. FIG. 2 illustrates a side view of the example of the pH sensor 102. The pH sensor 102 may include a working tag 104 and a reference tag 106, also collectively refer to as sensor tags, positioned on a substrate 108. The resonant frequency of the reference tag 106 may be different than the working tag 104. As described below, it may be preferable for the reference tag 106 to have a higher resonant frequency.

The reference tag 106 and a portion of the working tag 104 may be covered with a passivation layer 110 for insulation. The passivation layer may provide a water proof or hydrophobic layer. For example, the passivation layer 110 may be silicone or the like. The pH sensor 102 may further include a water soluble layer 112 and pH sensitive layer 114. The water-soluble layer 112 may be applied to the exposed portion of the working tag 104, and the pH sensitive layer 114 may be applied on top of the water soluble layer 112.

During operation, the pH sensitive layer 114 may dissolve when pH rises above a threshold level, such as 6.8, which indicates spoilage. After the pH sensitive layer 114 dissolves, the water soluble layer 112 may also dissolve, thus changing the resonant frequency of the working tag. The sensing tag and reference tag may be positioned on the sensor in parallel. Changes in the resonant peak are driven by the dissolution of a pH sensitive bilayer coated onto a UHF sensing resonator. Large-area spoilage detection of packaged meat products may be measured wirelessly by taking advantage of the far-field communication enabled by the resonator design. UHF resonators may be manufactured through a scalable laser-etching process, which resulted in tags with high sensitivity to changes in the dielectric constant of their immediate environment and increased surface energy, resulting in a far-field sensing platform with improved adhesion properties.

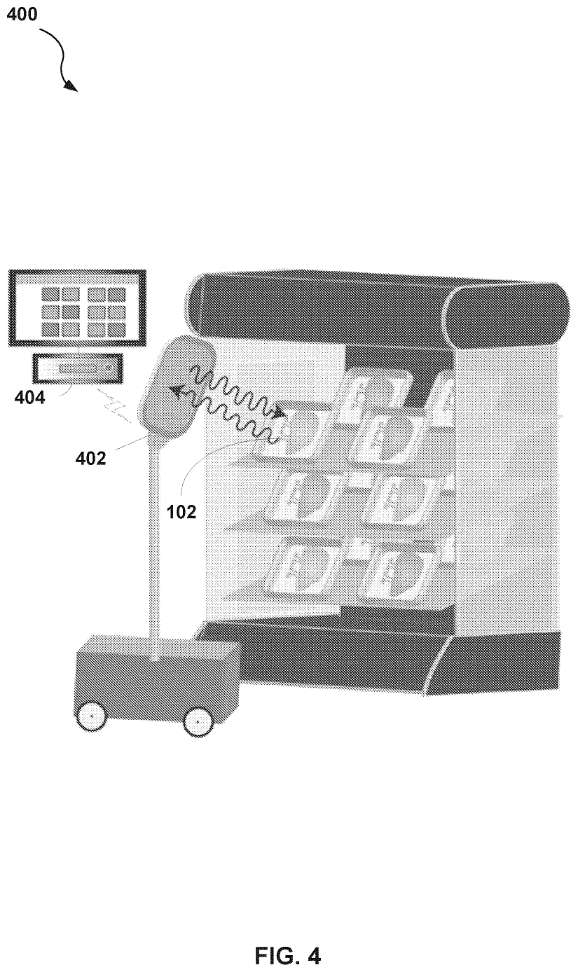

FIG. 3 illustrates an example of a working principle of the pH sensor. Tags are placed within packaged meat products, between the meat and absorbing pad, or directly touching the meat. Changes to a pH-sensitive coating occur upon spoilage and are captured as a distinct shift in resonant peak. The reference tag provides a stable peak for relative change calculations. The ability of far-field communication enables large area interrogation, making the sensor tag ideal for use in large food stores.

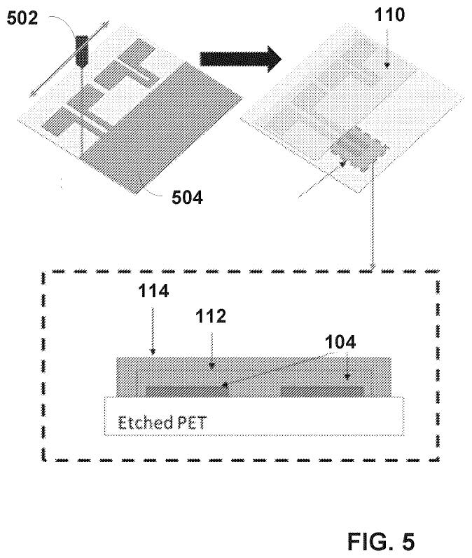

FIG. 4 illustrates an example of a system 300 for detecting food spoilage. The system 400 may include a scanner 402. The scanner 402 operates by interrogating the pH sensor 102 with a signal and receiving a response. The system 400 may further include a device 404. The device 404 may include a hardware processor which communicates with the scanner. The hardware processor may execute instructions which cause receive signals from the scanner. The processor may measure a first resonant frequency of the working tag and a second resonant frequency of the reference tag. The processor may determine, based on the first resonant frequency and the second remnant frequency, a food spoilage event. The processor may output the food spoilage event. For example, the processor may generate a graphical user interface and/or cause a graphical user interface to be displayed on a display interface. The graphical user interface may indicate which food packages are safe and which have gone bad based on interrogation of the pH sensor.

### Reagents and Materials

In various experimentation, aluminum coated PET sheets of thickness 0.254 mm were used for the substrate later. The PET sheets were non-tearing, heat resistant, and chemically resistant. Eudragit S100 was utilized for the pH sensitive layer. Polyvinyl alcohol (PVA, MW: 9,000-10,000, 80% hydrolyzed) was utilized for the water-soluble layer. Chicken breasts used in in-vitro experiments were acquired to conduct the experimentation.

The PVA solution used in various experiments may be prepared by dissolving 70 wt % PVA powder in DI water and allowed to stir at 40° C. overnight using a stir bar and stirring plate. The S100 solution may be prepared by dissolving 20 wt % S100 powder in an IPA/Acetone (60/40) mixture and sonicated for 30 minutes at 2000 RPM.

### Fabrication of Flexible pH Sensing Tag

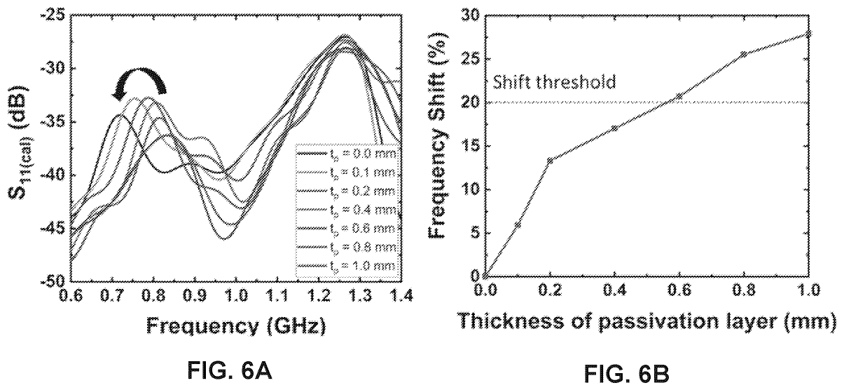

FIG. 5 illustrates a manufacturing process for the pH sensor. The working and reference tags may be fabricated using a laser 502 according to a laser etching directed at a metalized sheet, such as a metalized PET 504. The tags may be subsequently coated with the water soluble coating 112 (e.g. PVA) and the pH-sensitive coating 114 (e.g. S100) and passivated with a heat-cure silicone adhesive.

By way of example, the sensor tags may be manufactured through a laser etching process using a Universal Laser PLS6MW laser engraver equipped with a fiber source (1.06 μm) by optimizing parameters to 16 W power and 1.2 m/s speed (FIG. 4a). The working tag and reference tag may be patterned through a single laser-etching step on the same metalized PET substrate. The high selectivity of the fiber laser to AI at the optimized settings of power and speed kept the underlying PET sheet intact from the heat throughout the process. The active area of laser-ablated working tags may be coated with the pH-sensitive bilayer including a 500-micron PVA layer followed by a 100-micron S100 enteric coating.

### Sensor Tag Design

In various experimentation, the design of the working tag was optimized for high sensitivity to changes in coating thickness and resonant frequency. An operating frequency range of 0.5-1.5 GHz was chosen to prevent the attenuation of RF signals above 2 GHz due to the skin depth limitations imposed by the highly moist nature of the food product and to restrict the size of the tags to fit within the food package as frequencies lower than 0.5 GHz would increase the dimensions of the tags. Since the sensor tags can experience high attenuation if placed underneath the food product, it was necessary to isolate the sensing area from the antenna area. A dipole antenna was used for the sensor tag as it has an antenna area that is separate from the sensing area. The horizontal section of the dipole design acts as the antenna area whereas the feed point of the vertical section, being the most sensitive region to impedance variations, acts as the sensing area. Once the dimensions of the dipole design were fixed, the working tag's response to changes in coating thickness and optimum thickness were analyzed through simulations. The reference tag was designed as a dipole antenna with the feed points shorted with each other such that its resonant frequency is higher than that of the working tag. Since the resonant frequency of the working tag is expected to reduce with the degradation of the coating, having a higher resonant frequency for the reference tag prevents overlap between the resonant peaks of the working tag and the reference tag in the frequency spectrum. Although the working tag and the reference tag were on the same substrate, the reference tag was physically separated from the working tag by 15 mm in order to prevent cross coupling.

### Surface Characterization

In various experimentation, scanning electron microscopy (SEM) imaging of the surface and cross-section of pristine, laser-etched, and coated PET films was performed using a Hitachi S-4800 SEM at an excitation voltage of 15 keV. Changes in surface chemical composition and structure due to laser-etching process were characterized through Fourier Transform Infrared (FTIR) spectroscopy using a PerkinElmer Spectrum 100 FTIR from 500 cm−1 to 4000 cm−1. Changes in surface static, advancing, and receding angle were measured using a DSA25 Drop Shape Analyzer from KRÜSS® by monitoring the contact angle between a 10 μL droplet and the film surface as the stage tilted until the droplet moved. The adhesion strength between untreated, plasma treated, and laser-etched PET sheets and PVA were tested through a modified Scratch-Tape test. The PVA coating was doped with methylene blue for improved contrast and “scratched” into a 1×1 mm grid with 200×200 μm resolution. The percent loss was quantified based on the number of squares removed from the grid after peeling the surface using duct tape at a 90° angle. The force required to peel the tape was quantified using an ADMET MTEST Quattro tensile test analyzer. The same procedure was followed to measure the adhesion strength between S100 and PVA.

### Solubility Characterization

Colorimetric solubility characterization of PVA, S100, and the S100+PVA bilayer was performed using a CLARIOstar microplate reader from BMG Labtech at a fixed wavelength of 666 nm. Films were doped with 10 wt % methylene blue and cut into 1 cm disks. A calibration curve for each coating in each buffer (5.0, 6.0, 7.0, 8.0, and 9.0) was derived by analyzing the absorption intensity of different concentrations of methylene blue dissolved in DI water, equivalent to 1 disk=25% dissolved. Four disks were placed in 100 mL of clear pH buffer solution, stored at 7° C., and allowed to dissolve for 10 days. For characterization of the S100+PVA bilayer, the PVA coating was doped with methylene blue.

### RF Characterization of Sensor Tags

The RF characterization of the sensor tags was performed with a reader that comprised of an Agilent Vector Network Analyzer (VNA) connected to a horn antenna. The VNA can operate in the frequency range of 100 MHz to 8 GHz and can output a maximum power of 13 dBm. The horn antenna can operate in the frequency range of 1 MHz to 12 GHz and can provide a gain of 9 dB. The reader setup was used for measuring the S11 characteristics of the sensor tags in solubility experiments with buffer solution and packaged chicken.

Far-field solubility characterizations of coated tags were performed by placing sensing tags in 200 mL clear mixing cups containing 120 mL of buffer solution and kept at 7° C. The sensor tags were placed at a reading distance of 25 cm from the reader. The frequency of the VNA was swept from 0.5 GHz to 2 GHz with a constant output power of 0 dBm and the changes in the resonant peaks were measured in the S11 characteristics over a 10-day period.

### Sensor Characterization With Chicken Samples

In various experimentation, the sensor characterization was performed by placing coated sensing tags and passivated reference tags within packaged chicken samples and kept at 7° C. Changes in the resonant peak due to spoilage were measured over a 10-day period using the RF reader. Spoilage was achieved by keeping experimental samples in an incubator at 37° C. for 10 hours prior to the experiment. Control samples were kept refrigerated throughout the experiment. Changes in surface pH were measured using an IntelliCAL®PHC729 pH probe to confirm spoilage. The surface pH of both control and experimental samples was measured prior to the start of the experiment. Traditional foam and clear plastic wrap were used as a food package.

### Results and Discussion

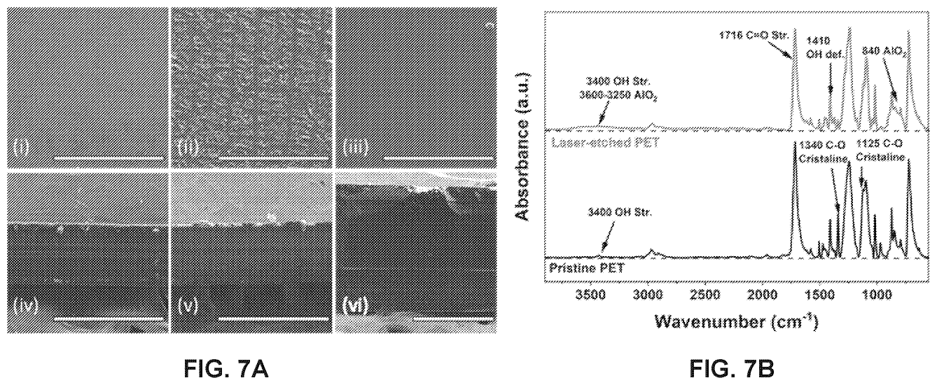

Simulations—FIG. 6A illustrates a calibrated S₁₁ as a function of frequency for various thicknesses of the passivation layer (tₚ) demonstrating a decrease in the resonant frequency with tₚ. FIG. 6B illustrates optimization of the thickness of the passivation layer demonstrating 0.6 mm as the minimum thickness to obtain a shift threshold of 20%.

In an experimental setup, a VNA connected to a horn antenna measured the S11 characteristics from both working and reference tags simultaneously. CST microwave studio was used to simulate the reader presented in the experimental setup along with the sensor tag to find the optimum thickness of the coating on the working tag required for the solubility test. The thickness of the coating, denoted as tₚ, was varied from 0 mm to 1 mm such that a finite value of tₚ indicated the thickness before dissolution and tₚ=0 mm indicated complete dissolution. Simulation results modelling the shift in the working tag's resonant peak due to coating dissolution suggested a shift towards lower frequencies as well as a small decrease in intensity as the thickness was decreased. While the resonant frequency of the working tag changed from 0.841 GHz to 0.724 GHz as tₚ was varied from 1 mm to 0 mm, the resonant peaks of the reference tags remained at 1.26 GHz with negligible variations with respect to tp. The relative shift obtained when the thickness of the coating was varied from a finite initial value to zero indicating complete dissolution was plotted in FIG. 6A. Optimization results show that the film thickness required to reach a minimum 20% shift in the resonant peak is 0.6 mm (FIG. 6B). A minimum shift threshold of 20% is required to ensure that the relative shift obtained is well above the noise margin.

In various experimentation the ETS-Lindgren horn antenna was placed 25 cm away from the coated tags in an anechoic chamber. Although S100 is the material that provides a pH-triggered dissolution, it is not possible to reach the required 0.4 mm minimum thickness needed to achieve a 10% shift in the resonant peak. Eudragit S100 is a commercial resin designed for drug delivery applications in the digestive system as a thin enteric coating with rapid dissolution above a pH threshold of 6.8. Despite its rapid solubility above the pH threshold and excellent properties as an enteric coating, it is difficult to develop a thick coating using S100 alone. This is mostly due to the highly crystalline structure of the acrylic backbone of S100, which makes the material brittle and thick films difficult to achieve. In order to reach the required thickness, PVA with relatively low molecular weight and percent hydrolyzation was used as an intermediary layer, which can quickly dissolve in aqueous media across all pH values. Given its low molecular weight, achieving thick, flexible, and water-soluble PVA films is less cumbersome compared to a thick S100 coating.

### Surface Characterizations

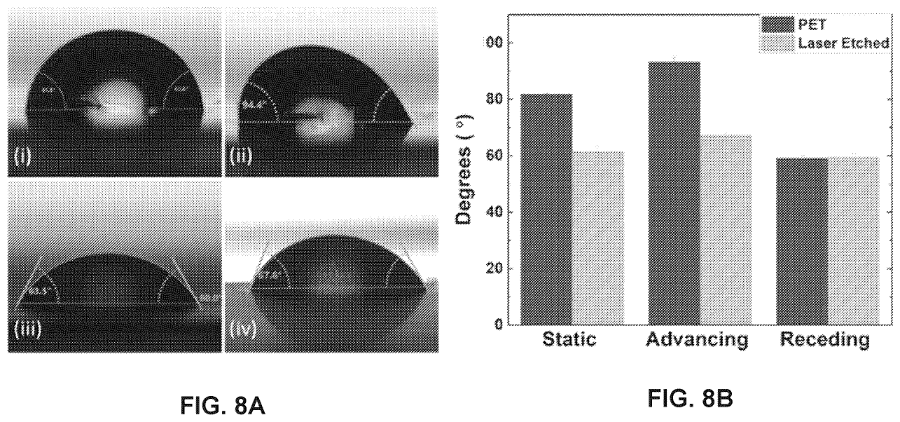

FIG. 7A-B illustrates SEM imaging and FTIR characterization. FIG. 7A illustrates surface SEM images (i-iii) of metallized PET, laser-etched PET, and pH-sensitive coated tag, as well as cross-sectional SEM images (i-iv) of metallized PET, laser-etched PET, and pH-sensitive coated tag. FIG. 7B illustrates results from FTIR analysis displaying increase in OH content, AlO2 deposition, and amorphization of surface of PET after laser-etching.

SEM images of the surface and cross-section of metalized, laser-etched, and coated PET samples are shown in FIG. 7A. Surface images show the effect of laser etching on the metallic coating of the PET. FIG. 7Ai shows a smooth aluminum coating, the product of Chemical Vapor Deposition (CVD), on the PET surface. Upon laser etching, the aluminum nanoparticles evaporate off the film as they absorb energy from the focused laser-beam. This results in immediate removal of the aluminum coating and exposure of the underlying PET sheet. This technique has been used recently as an alternative to traditional lithography methods as a more scalable, rapid, and environmentally friendly method of manufacturing flexible electronics. It is advantageous compared to analog printing techniques, which require the use of conductive inks and pastes plus a drying/sintering step, as a more rapid method, yielding traces with competing conductivity. Through this process, there is a dual effect of removal of the aluminum coating from the PET as well as modification of the underlying surface. As shown in FIG. 7Aii, an increase in surface roughness occurred as the heat transferred from the aluminum onto the PET, resulting in a highly ordered topography. These newly formed structures and consequent increase in roughness are expected to modify the surface interactions between PET and the following PVA coating. FIG. 7Aiii shows the smooth surface of the PVA layer on the ablated PET. Cross-sectional images provide a clearer description of the effect of the laser etching process and the magnitude of the subsequent coating. As seen in FIG. 7Aiv and FIG. 7Av, for the metalized and laser-etched PET sheet respectively, the increase in surface roughness given newly formed topography following laser etching is in the order of a few nanometers. These structures are dwarfed in comparison to the PVA coating, which is 0.5 mm in size (FIG. 7Avi). The cross-sectional images of the PVA coating show a thick, smooth, and dense film which provides the necessary thickness to achieve the targeted shift in the resonant peak.

FTIR analysis of pristine and laser-etched PET samples shows the effect of the laser etching process on the metalized PET sheets in FIG. 7B. The peaks at 3400 cm−1 and 1716 cm−1, present on both profiles, are associated with OH and C═O stretching. The presence of OH functional groups on the surface of laser-etched samples is positive in terms of promoting adhesion between the PVA coating and PET. The lack of change in intensity of the 1716 cm−1 peak is also positive as it suggests a lack of photodegradation of ester bonds as the PET is exposed to the fiber laser. Previous studies involving laser treatment of PET using a CO2 source show there is a decrease in this peak associated with photodegradation as the polymer sheet absorbs the energy emitted from the laser at the lower wavelength. These results suggest that any modification to the surface of the PET samples happening through the etching process is due to heat transferring from the deposited aluminum onto the polymer surface and not due to energy absorption by the PET. The peaks at 1340 cm−1 and 1125 cm−1 on the pristine sample are characteristic of crystalline regions in PET. A decrease in intensity at these wavelengths, as seen on the laser-etched profile, suggests that there is an amorphization of the PET as it is ablated. This change occurs, similarly to the change in topography, due to heat transfer during the etching process. Interestingly, the appearance of a “broad hump” in the range of 3600-3250 cm−1 and the appearance of a peak at 840 cm−1 on the laser-etched profile suggests an increase in the concentration of aluminum oxide (AlO2) functional groups on the PET surface as a result of the etching process. Aluminum oxide is generated as the deposited aluminum nanoparticles on the PET surface rapidly absorb energy from the laser source in the air. Even though most of the aluminum is evaporated, a small percentage of aluminum remains on the surface interacting with available oxygen in the air at high temperatures within the instant it is exposed to the laser, resulting in AlO2 embedded onto the surface. AlO2 has several advantages within the scope of this application, providing improved adhesive properties between hydrogen-rich polymers and the PET surface. Further, metal oxides like those derived from aluminum and its conjugates, are of special interest in terms of their antimicrobial properties and applications in nano-scaled biosensor development. Uniform functionalization of the metalized PET with AlO¬2 groups in a 1-step process can be achieved through the laser etching method described.

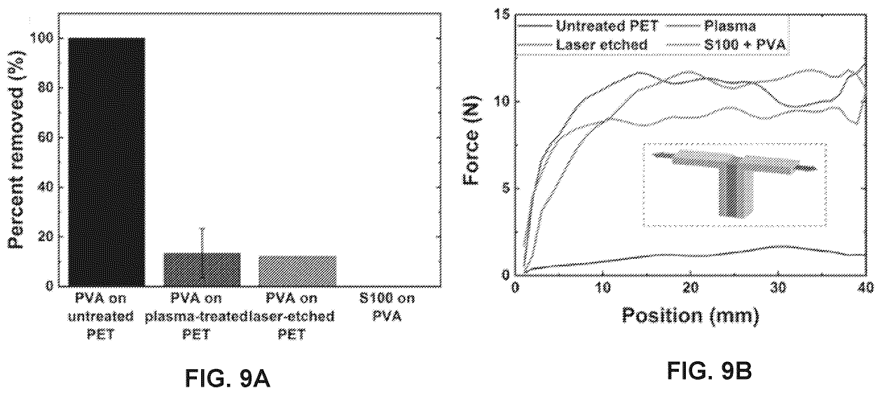

FIG. 8A-B illustrates results from contact angle experiment. FIG. 8A illustrates images of droplet on untreated PET samples during (i) static and (ii) dynamic contact angle measurement, as well as images of droplet on laser-etched PET during (iii) static and (iv) dynamic contact angle measurement. FIG. 8B illustrates a summary plot showing changes in static, advancing, and receding contact angle due to laser-etching process. The static and advancing angle are reduced, while the receding angle remains unaffected.

Static and dynamic surface contact angle (CA) measurements of pristine and laser-etched PET samples are shown in FIG. 8. FIG. 8Ai and FIG. 8Aii show images of a water droplet on pristine PET during static and dynamic CA measurements, respectively. FIG. 8Aili and FIG. 8Aiv show images of a water droplet on laser-etched PET during static and dynamic CA measurements, respectively. A summary plot of the measured CA is shown in FIG. 8B. Visually, a clear distinction between pristine and laser-etched PET samples can be observed, as the droplet on laser-etched samples has a larger spread and lower static CA during, which indicates higher wettability. Results from dynamic CA measurements show an average advancing angle of 94° and a receding angle of 59° for pristine PET samples, and an average advancing angle of 64° and a receding angle of 59° for laser-etched PET samples. A decrease in advancing CA through the laser-etching process suggests the removal of hydrophobic species off the surface of the PET which results in higher wettability. Simultaneously, a decrease in hysteresis through the laser-etching process suggests that it not only affects the wettability of the surface, but also improves its homogeneity. This process presents a dual effect by modifying both the chemical and physical properties of the PET surface by increasing the wettability through the formation of functional groups, such as AlO2, and homogenization of the surface through the formation of a highly ordered topography.

### Enhanced Adhesive Properties

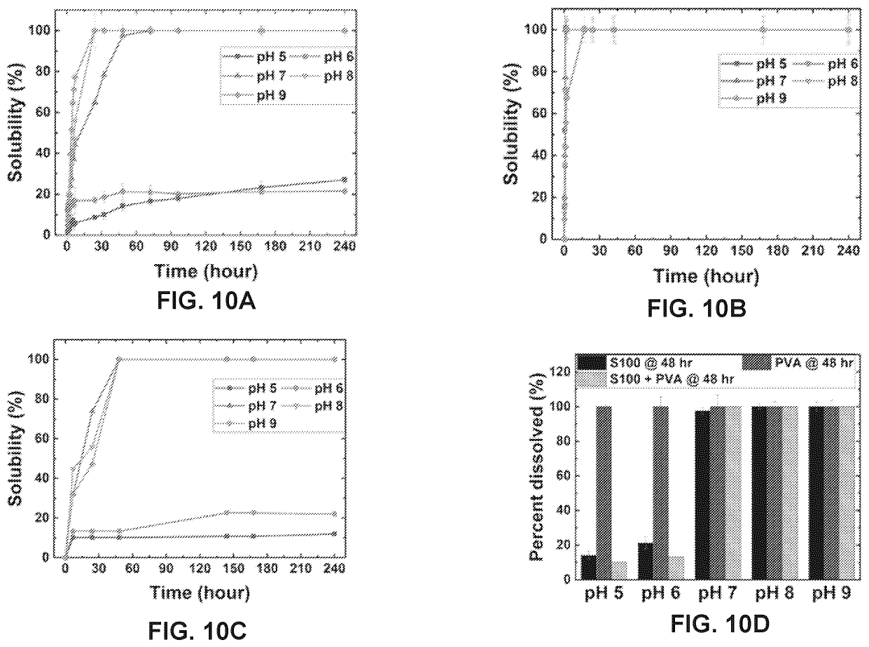

FIG. 9A-B illustrates results from scratch tape test to determine changes in adhesion. FIG. 9A illustrates a summary plot showing percent removed off each surface. FIG. 9B illustrates results from force analysis during peel test.

The effect of the laser-etching process on the adhesive properties of the PET surface was characterized through a modified scratch tape test. Three conditions were tested: pristine PET, laser-etched PET, and plasma treated PET. The adhesive strength between these surfaces and the intermediary PVA layer was tested. The effect of plasma treatment was explored as a referent given its increased use as a method of improving the wettability and adhesive properties of several polymers, including PET.

FIG. 9B shows a summary plot quantifying the percent removed in each condition. The laser-etching process results in less than 20% PVA removed, an improvement from complete removal off untreated samples, and similar to plasma treatment. There is 0% removal of S100 off PVA. Simultaneously, the tensile force profile as the tape was removed was recorded and reported in FIG. 9C. The results from the force measurements are consistent with the percent removed in each condition. For untreated samples, a relatively low force (<2 N) is needed to peel off the tape as the PVA coating is readily removed. For laser-etched and plasma treated samples, a high force of 12 N is needed.

The results suggest that the chemical and physical modifications of the PET surface, product of laser-etching, lead to an increase in the adhesive properties between PET and hydrogen containing polymers such as PVA. This increase in adhesive strength occurs with the formation of functional groups on the surface of PET, such as AlO2 and OH, which provide anchoring sites for maximized bonding. This effect is similar to the enhanced adhesion through plasma treatment, with the benefit of being achieved in a single step during the manufacturing process without further modification. These results also demonstrate the excellent bonding between S100 and PVA, suggesting the adequacy of using PVA as an intermediary layer. Although PVA is not soluble in acetone and IPA, the main solvents in the S100 solution, swelling due to exposure to acetone could explain the strong adhesion between these two polymers. Exposure to acetone induces an increase in surface roughness of the PVA layer, thus creating sites for physisorption.

### Solubility Characterizations in Different pH Buffers

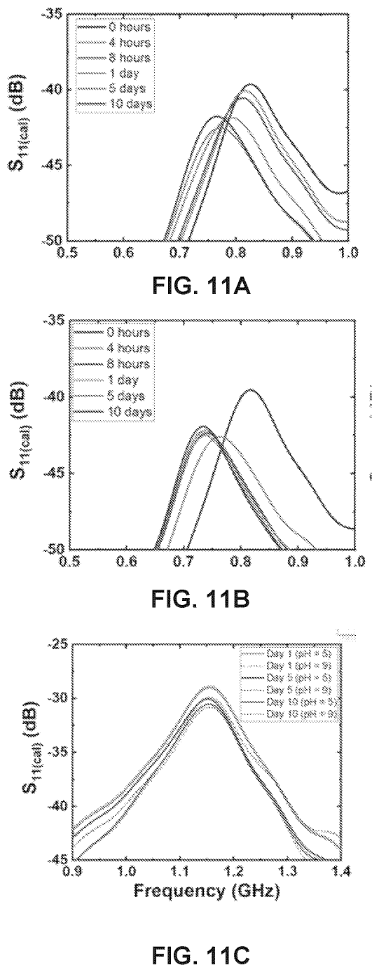

FIG. 10A-D illustrates results from solubility characterizations. FIG. 10A illustrates solubility characterization of S100 in pH 5, 6, 7, 8, and 9. FIG. 10B illustrates solubility characterization of PVA in pH 5, 6, 7, 8, and 9. FIG. 10C illustrate solubility characterization of S100+PVA in pH 5, 6, 7, 8, and 9. FIG. 10D illustrates a summary plot of solubility test across all buffers for all three samples after 48 hours.

Results from solubility characterizations measuring the percent dissolved as a function of die release in different buffer solutions are shown in FIG. 10. The solubility of individual S100 and PVA layers was tested, as well as the solubility of the S100+PVA bilayer. FIG. 10A shows the solubility profile of S100 in different buffers over a period of 10 days, stored at 7° C. Below the pH threshold of 6.8, S100 shows limited dissolution, stabilizing at 20% dissolved after 10 hours. This slight dissolution could be a product of excess methylene blue on the surface of the films or water absorption in aqueous buffer solutions which can allow for some die leaching. Above a pH of 6.8, S100 rapidly dissolves, reaching 60% dissolution in pH 7 and 100% dissolution in pH 8 and pH 9 buffers in 24 hours. In the case of PVA, shown in FIG. 10B, the dissolution is considerably faster compared to S100. Complete dissolution is achieved within 10 hours across all pH values. Given the intended application, PVA powder with low molecular weight and low percent hydrolyzation is used. The low molecular weight allowed for a viscous solution containing a high percent of the polymer, which in turn facilitated the casting of thick films and reduced drying time considerably. Simultaneously, the low molecular weight and low hydrolyzation percent enabled faster dissolution in aqueous media. FIG. 10C shows the solubility profile of the S100+PVA bilayer. Clearly, similarities exist between the solubility of the bilayer and the solubility of S100 only. There is a slight dissolution in pH 5 and pH 6 buffer reaching a maximum of 15% in the first 48 hours and rising to 20% after 6 days in pH 6. Above S100's pH threshold, the bilayer dissolves steadily over time, with some delay compared to S100 only. For the characterization of S100+PVA, methylene blue was only added to PVA therefore, the observed delay is due to S100 dissolution. FIG. 10D shows a summary plot for all three conditions in each buffer after 48 hours. These results demonstrate the stability of S100 as a pH-sensitive protective layer, which only allows for the complete dissolution of the S100+PVA coating above the pH threshold. Both S100 and S100+PVA show significant dissolution only above pH 7, while PVA rapidly dissolves across all pH values.

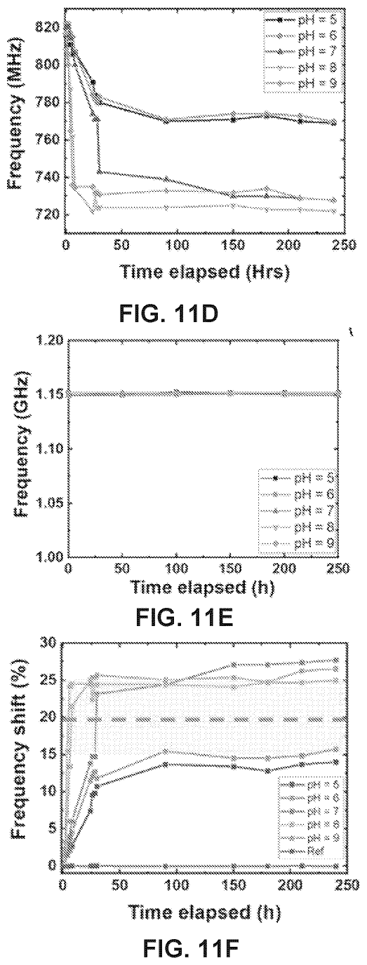

FIGS. 11A-H illustrates results for solubility characterizations in pH buffers measured as changes in the resonant peak of both working and reference tags. FIG. 11A illustrates a photograph of experimental setup showing a horn antenna placed 20 cm away from the sensor comprising both working and reference tags. FIG. 11B illustrates an image of working tags within clear containers with each buffer solution. FIG. 11C illustrates resonant peak of working tag in buffer pH 5 over 10-day period. FIG. 11D illustrates resonant peak of working tag in buffer pH 9 over 10-day period. FIG. 11E illustrates resonant peak of reference tag in buffers of pH 5 and pH 9. FIG. 11F illustrates changes in peak frequency of working tags in buffers pH 5, 6, 7, 8, and 9 over time. FIG. 11G illustrates changes in peak frequency of reference tags in buffers pH 5, 6, 7, 8, and 9 over time. FIG. 11H illustrates percent frequency shift of working and reference tags, showing the working tag reaching a maximum frequency shift of 15% (5% below threshold) below pH 7, while it reached a maximum frequency shift of 25% above pH 7 (5% above threshold) within first 100 hours.

FIGS. 11A-B, show the shift in the resonant peak of working tags in buffers of pH 5 and pH 9 over time, respectively. This is caused by a change in the effective dielectric constant in the vicinity of the tag from εᵣ₍ᵢₙᵢₜᵢₐₗ₎(=2) to ε_{r(final)}(2<ε_{r(final)}≤80) which depends on the amount of water seepage closer to the tag with dissolution or water uptake of the bilayer. Consequently, in both conditions, the resonant peaks shift towards lower frequencies as the bilayer is exposed to the media. In pH 5 buffer, there is a mild shift from 820 MHz to 780 MHz, which can be attributed to interactions between the tag's electric field and slight water uptake by the bilayer causing the ε_{r(final)} to increase from 2 to a value much less than 80. In contrast, in pH 9 buffer, complete dissolution of the bilayer coating leads to a significant amount of water contacting the tag causing a bigger change in the effective dielectric constant (ε_{r(final)}≈80) surrounding the tag resulting in a larger shift in the resonant peak, from 820 MHz to 720 MHz. FIG. 11C shows changes in the resonant peak of reference tags in pH 5 and pH 9 buffers over time. Since the reference tags were shorted at the vertical end, it displayed a distinct resonant peak, unaltered by the changes in the sensing area of the working tags. The presence of reference tags helped in the easier differentiation and simultaneous readout of resonant frequencies. This was shown in FIG. 11C, where the initial frequency in both pH buffers is 1.18 MHz and as expected, a negligible difference was observed in the resonant peaks of the reference tags in pH 5 and pH 9 buffers with the progress of time. FIG. 11D shows a summary plot of the changes in the resonant peak over time across all pH buffers (5-9). Below the pH threshold, a slight shift was observed, similar to the one observed in solubility characterizations based on die release. This serves as further evidence that water absorption leads to an apparent dissolution of 20% in characterizations utilizing methylene blue and to a shift in the resonant peak in characterizations using the horn antenna. Above the threshold, a larger shift was observed. In pH 7 buffer, a step change can be observed at the 25-hour mark. Due to this buffer's proximity to the pH threshold, there is a transition from a shift in resonant peak due to water absorption towards a dissolution-dominated shift. As observed earlier, dissolution profiles for S100+PVA show a delay given S100's solubility behavior in this buffer. The dissolution of S100 in this buffer is slower compared to PVA alone. While S100 goes through the water absorption step before dissolving, the dissolution of PVA is almost immediate, which results in the observed step change. In the case of pH 8 and pH 9 buffers, the S100 layer goes through the water absorption step quickly, leading to a rapid shift in the resonant peak with a less apparent step change. FIG. 11E shows the changes in the resonant peak of reference tags across all buffers over time. These results show the stability of the passivated reference tag in different buffers, as there is no apparent shift. The silicone passivation does not go through the water absorption step, which leads to a stable resonant peak over time. FIG. 11F shows the percent frequency shift of both working and reference tags across all buffers over time. Above the pH threshold, a maximum shift of ~25% was achieved after 48 hours, while below the threshold, the percent frequency shift reached a plateau at 15%. Since 20% is the mean value of the maximum shifts below pH 7 and above pH 7, defining the shift threshold at 20% provides an optimum error deviation from the critical final values of the percentage frequency shift for evaluating food spoilage. This is corroborated by the simulation results where coating the working tags with a film of thickness above 0.6 mm provided a percentage frequency shift of above 20% as a result of complete dissolution. The reference tag shift remains at a 0% shift over time across all pH buffers. These results provide clear evidence for the stability of this sensing system in different buffers and validate the simulation results. By utilizing a thick coating, comprising S100 and PVA, the minimum required shift of 20% was achieved only when exposed to buffers above the threshold. At pH 7 and above, the S100 coating fully dissolved, exposing the thick PVA intermediary layer, and only then was the 20% shift observed. Below pH 7, only a 15% shift was observed, which is well below the shift threshold that indicated incomplete dissolution.

### Conclusion

In this work, a flexible, battery-less, chipless pH sensor was manufactured through a scalable laser-etching process for applications in large area detection of spoilage of packaged meat products. The sensor utilized a sensing mechanism based on shifts in the resonant peak as a pH-sensitive bilayer, coated onto a laser-etched dipole resonator, was exposed to high pH values indicative of spoilage. The laser-etching process had multiple effects on the properties of PET, including enhanced wettability and adhesion, formation of highly ordered microstructure, and functionalization with metal nanoparticles, in a one-step process. These enhanced properties resulted in improved adhesive strength between PET and PVA, only achievable through additional treatments such as salinization and plasma treatment. The high solubility of the thick PVA intermediary layer and the precise sensitivity of S100 to changes in pH resulted in a highly sensitive sensor capable of detecting changes in pH even within standard refrigerated conditions at 7° C., showing 100% dissolution within 24 hours in aqueous pH buffer. A far-field solubility test of the sensors was carried out using a wireless readout unit to identify the spoilage threshold at a resonant frequency shift of 20%. In experiments with packaged chicken samples, the sensor system was capable of detecting spoilage wirelessly within 3 days of contact with spoiled chicken showing shifts in the resonant frequency of 25% and up to 35%, while also remaining stable in contact with fresh samples showing shifts ~5%.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

While various embodiments have been described, it will be apparent to those of ordinary skill in the art that many more embodiments and implementations are possible. Accordingly, the embodiments described herein are examples, not the only possible embodiments and implementations.

What is claimed is:

1. A pH sensor, comprising:
a substrate;
a working tag on the substrate, the working tag having a first resonant frequency;
a reference tag on the substrate, the reference tag having a second resonant frequency;
a first layer in contact with a portion of the working tag and not the second tag, the first layer being water soluble; and
a second layer positioned on top of the first layer, the second layer configured to dissolve when exposed to an environment having a pH level higher than a threshold.

2. The pH sensor of claim 2, wherein the reference tag is configured to change resonant frequencies in response to the dissolution of the first layer.

3. The pH sensor of claim 1, wherein the reference tag is aluminum.

4. The pH Sensor of claim 1, wherein the first layer comprises Polyvinyl acetate (PVA).

5. The pH Sensor of claim 1, wherein the second layer comprises s100.

6. The pH sensor of claim 1, further comprising a passivation layer positioned on top of the reference tag and a second portion of the working tag.

7. A method of manufacturing a pH sensor comprising:
etching a reference tag and a working tag onto a metallized substrate;
covering the reference tag and a first portion of the working tag with a passivation layer;
applying a first layer to a second portion of the working tag, the first layer the first layer being water soluble; and
applying a second layer on top of the first layer, the second layer configured to dissolve in response to exposure to an environment with a pH greater than a threshold value.

8. The method of claim 7, wherein the etching is performed via laser etching.

9. The method of claim 7, wherein the reference tag is aluminum.

10. The method of claim 7, wherein the first layer comprises Polyvinyl acetate (PVA).

11. The method of claim 7, wherein the second layer comprises s100.

12. A food package comprising:
a pH sensor, a substrate, a working tag and reference tag on the substrate, the working tag having a first resonant frequency, the reference tag having a second resonant frequency, a portion of the working tag being covered with a first layer and the first layer being covered by a second layer, wherein the first layer is water soluble and the second layer is configured to dissolve when exposed to content of the food package having a pH level higher than a threshold pH level.

13. The food package of claim 1, wherein the content comprises a perishable good.

14. The food package of claim 1, wherein the content further comprises an absorbent pad, wherein the absorbent pad is positioned between the perishable good and pH sensor.

15. The food package of claim 12, wherein the reference tag is configured to change resonant frequencies in response to the dissolution of the first layer.

16. The food package of claim 12, wherein the reference tag is aluminum.

17. The food package of claim 12, wherein the first layer comprises Polyvinyl acetate (PVA).

18. The food package of claim 12, wherein the second layer comprises s100.

19. The food package of claim 12, further comprising a passivation layer positioned on top of the reference tag and a second portion of the working tag.
